(19) United States
(12) Patent Application Publication
(10) Pub. No.: US 2017/0333250 A1
(43) Pub. Date: Nov. 23, 2017

(54) MEDICAMENT DISPENSER

(71) Applicant: Infield Medical, LLC, Morrisville, NC (US)

(72) Inventors: David Foshee, Morrisville, NC (US); Gretchen Willard, Morrisville, NC (US); Theodore Mosler, Morrisville, NC (US)

(21) Appl. No.: 15/523,907

(22) PCT Filed: Nov. 3, 2015
(86) PCT No.: PCT/US2015/058855

§ 371 Date: May 2, 2017

Related U.S. Application Data
(60) Provisional application No. 62/074,549, filed on Nov. 3, 2014.

(51) Int. Cl. A61F 9/00; A61J 1/20; A61J 1/14

(52) U.S. Cl. CPC A61F 9/0008; A61J 1/1406; A61J 1/201; A61J 1/2037

(57) ABSTRACT

A dispenser comprises a cylinder in fluid communication with a one-way valve and an inlet in fluid communication with the cylinder. A lumen and a flow control member deliver fluid from a container to the inlet. A piston draws fluid through the flow control member and inlet into the cylinder upon movement of the piston in a first direction, and expels the fluid through the one-way valve upon movement of the piston in a second direction. The dispenser may be activated by either a “dual-action” mode of operation where an activating member is moved to retract the piston and provide the stored energy and then an-actuator is depressed to release the stored energy to the piston or a “single-action” mode of operation where an actuator is depressed in a single direction which concurrently retracts the piston, provides the stored energy and then releases the stored energy to piston.

### TECHNICAL FIELD

This invention generally relates to a medicament dispenser with a piston and cylinder, valve assembly and actuator configured to receive a source of medicament for dispensing.

### BACKGROUND

Conventionally drops are applied to the eye or in the mouth with an dropper generally by squeezing a plastic container fitted with a tapered tube. This manner of application has a number of disadvantages such as the requirement to align the tapered tube precisely with the eye as well as from anticipation of the drop and involuntary blinking, both of which may result in the drop of medicament not being delivered to the ocular space. Furthermore, and particularly applicable to self-administration of eye drops, is the problem of uncertain dosage originally applied and the indeterminate fraction thereof that is squeezed out of the eye by reflex blinking.

Continued use of eye drops in certain instances is required for medical treatment. Thus, for example, in the treatment of glaucoma it is conventional to require the administration to the ocular space a number of times per day for life. The necessity of continued usage of eye drops as in the foregoing instance usually requires the drops to be self-administered and the present disclosure is particularly adapted to improve this application.

### SUMMARY

In a first embodiment, a dispensing device is provided comprising: a valve assembly comprising a cylinder in fluid communication with a one-way valve and an inlet in fluid communication with the cylinder, the valve assembly further comprising: a spike with at least one lumen; and at least one flow control member for delivering medicament from a medicament container to the inlet; a piston configured to draw an amount of fluid through the flow control member and inlet into the cylinder upon movement of the piston in a first direction, and to expel the amount of liquid through the one-way valve upon movement of the piston in a second direction; and an actuator assembly coupled to the piston for moving the piston in the first direction to a first state and for storing energy in a stored energy source and for releasing the stored energy to move the piston in the second direction.

In an aspect of the first embodiment, the flow control member provides one-way fluidic communication between the at least one lumen and the inlet.

In other aspect alone or in combination with any one of the previous aspects of the first embodiment, the spike has a second lumen fluidically coupled to a second flow control member providing one-way fluidic communication to a vent. In other aspect alone or in combination with any one of the previous aspects of the first embodiment, the stored energy source comprises a spring.

In other aspect alone or in combination with any one of the previous aspects of the first embodiment, movement of the piston in the first direction delivers a predetermined amount of medicament to the cylinder.

In other aspect alone or in combination with any one of the previous aspects of the first embodiment, movement of the piston in the first direction and the second direction is sequential. In other aspect alone or in combination with any one of the previous aspects of the first embodiment, the actuator assembly includes an actuator operatively connected to an activating member by a transmission member, the activating member being operatively connected to the piston for moving the piston in the first direction when the actuator is moved in a third direction. In other aspect alone or in combination with any one of the previous aspects of the first embodiment, the transmission member automatically releases the activating member upon continued movement of the activator in the third direction. In other aspect alone or in combination with any one of the previous aspects of the first embodiment, the one-way valve has a cracking pressure that is less than the force generated by the piston driven by the stored energy source.

In other aspect alone or in combination with any one of the previous aspects of the first embodiment, the spike pierces a penetrable septum of the medicament container.

In a second embodiment, a dispensing device is provided comprising: a valve assembly comprising a cylinder in fluid communication with a one-way valve and an inlet in fluid communication with the cylinder, the valve assembly further comprising a spike with a lumen and a flow control member for delivering medicament from a medicament container to the inlet; a piston configured to draw an amount of fluid through the flow control member and inlet into the cylinder upon movement of the piston in a first direction, and to expel the amount of liquid through the one-way valve upon movement of the piston in a second direction; and an actuator assembly coupled to the piston for moving the piston in the first direction to a first state and for storing energy in a stored energy source, an actuator for holding the piston in the first state and for releasing the piston to release the stored energy to move the piston in the second direction.

In an aspect of the second embodiment, the flow control member provides one-way fluidic communication between the lumen and the inlet. In other aspect alone or in combination with any one of the previous aspects of the second embodiment, the spike has a second lumen fluidically coupled to a second flow control member providing one-way fluidic communication to a vent.

In other aspect alone or in combination with any one of the previous aspects of the second embodiment, the stored energy source comprises a spring.

In other aspect alone or in combination with any one of the previous aspects of the second embodiment, movement of the piston in the first direction delivers a predetermined amount of medicament to the cylinder. In other aspect alone or in combination with any one of the previous aspects of the second embodiment, movement of the piston in the first direction and the second direction is sequential.

In other aspect alone or in combination with any one of the previous aspects of the second embodiment, the actuator assembly includes an activating member for moving the piston in the first direction. In other aspect alone or in combination with any one of the previous aspects of the second embodiment, the actuator engages the activating member to hold the activating member in the first state. In other aspect alone or in combination with any one of the previous aspects of the second embodiment, the one-way valve has a cracking pressure that is less than the force generated by the piston driven by the stored energy source.

In other aspect alone or in combination with any one of the previous aspects of the second embodiment, the spike pierces a penetrable septum of the medicament container.

In a third embodiment, the device of the first or the second embodiment is used for dispensing medicament to the eye of a mammal.

In a forth embodiment, a method of operating a dispensing device is provided comprising a valve assembly comprising a cylinder in fluid communication with a one-way valve and an inlet in fluid communication with the cylinder, the valve assembly further comprising: a spike having at least one lumen; and at least one flow control member for delivering medicament from a medicament container to the inlet, the method comprising: moving a piston in a first direction to draw an amount of fluid through the flow control member and inlet into the cylinder and for storing energy in a stored energy source, and releasing the energy from the stored energy source to move the piston in a second direction to expel the amount of liquid through the one-way valve.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A and 1B are perspective views of the eye medicament dispenser in accordance with an embodiment of the present disclosure.

FIGS. 2A-2F are, respectively, a top plan view, a first side view, a bottom plan view, a second side view, a front view, and a rear view, of the eye medicament dispenser of FIG. 1A, in accordance with the present disclosure.

FIG. 3 is an exploded view of a medicament container in accordance with the present disclosure.

FIG. 4 is a top plan view of the eye medicament dispenser of FIG. 1A showing sectional line 6-6 in accordance with the present disclosure.

FIG. 5 is an exploded view of the eye medicament dispenser of FIG. 1A in accordance with the present disclosure.

FIG. 6 is a sectional view along section 6-6 of the eye medicament dispenser of FIG. 4 in a first state, in accordance with the present disclosure.

FIG. 7A is an expanded sectional view of area 7A of FIG. 6 of the valve assembly of the eye medicament dispenser in accordance with the present disclosure.

FIG. 7B is an exploded view of the valve assembly of the eye medicament dispenser in accordance with the present disclosure.

FIG. 8 is a perspective view of an eye medicament dispenser in accordance with another embodiment of the present disclosure.

FIG. 9 is an exploded view of the eye medicament dispenser of FIG. 8A in accordance with the present disclosure.

FIGS. 10A and 10B are vertical sectional view showing the eye medicament dispenser of FIG. 8 in a first state and a second state, respectively, in accordance the present disclosure.

FIG. 10C is a horizontal sectional view, showing the eye medicament dispenser in the first state in accordance with the present disclosure.

FIG. 11 is a perspective view of the eye medicament dispenser of FIG. 1A with an alternative valve assembly embodiment in accordance with the present disclosure.

FIG. 12 is an expanded sectional view of area 15 of FIG. 11 of the valve assembly of the eye medicament dispenser in accordance with the present disclosure.

### DETAILED DESCRIPTION

An apparatus and method for applying medicament to the eye rapidly by urging an amount of fluid from a medicament container through an inlet into a cylinder via a flow control valve. The medicament is urged with sufficient velocity from the cylinder with a piston through a one-way valve to minimize delivery loss via blinking of the eye during self-administration. In one aspect of the present disclosure the present device is devoid of a pressurized air source.

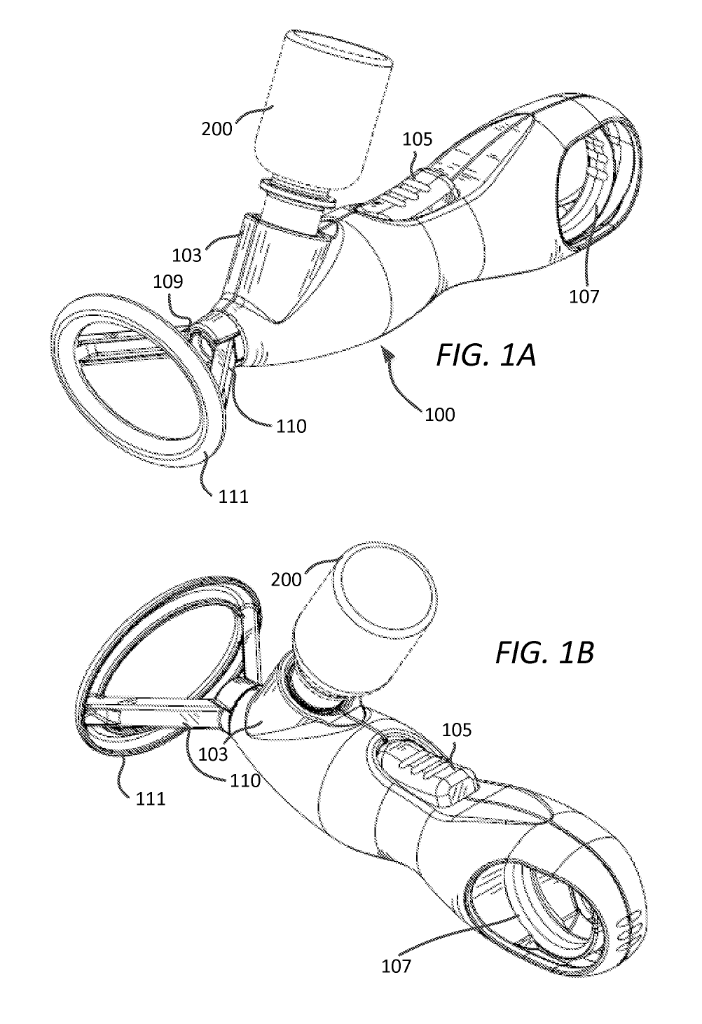

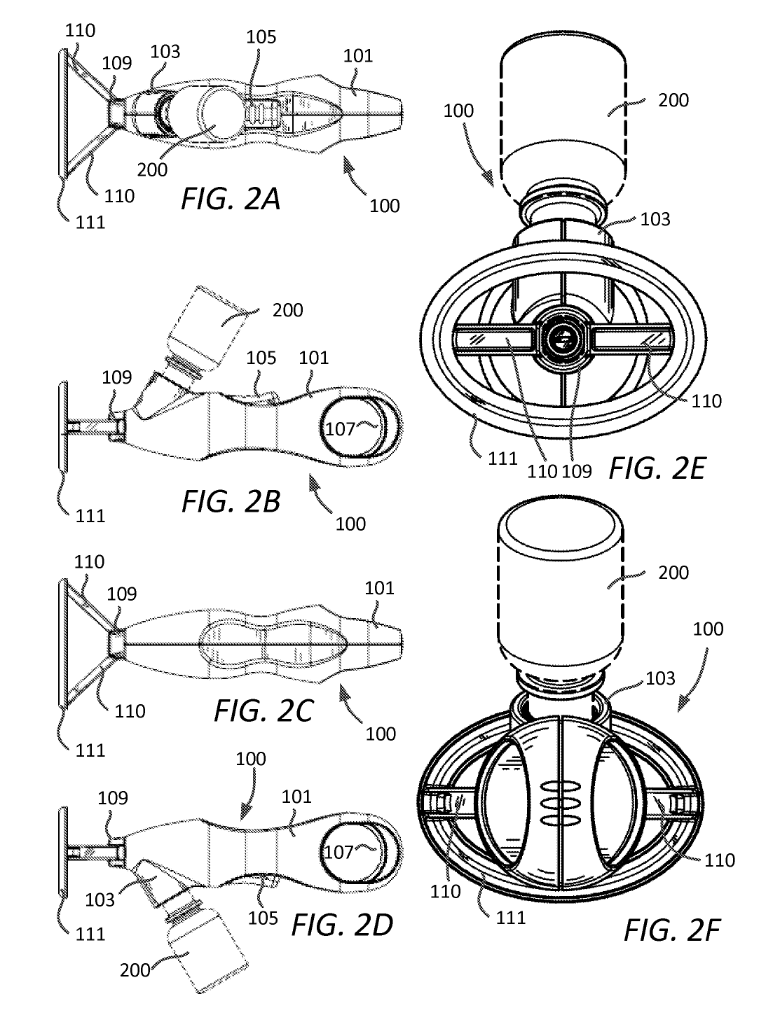

With reference to FIGS. 1A and 1B, dispenser 100 is shown comprising housing 101, including adapter 103 for receiving medicament vial 200, optional projection 111 with mounts 110 attached to flange 109 for coupling to housing 101 for locating and/or positioning the device in proximity to the user's eye, and user-actuator button 105 operably coupled to activating member 107. Activating member 107 comprises an accessible ring that may be engaged by a user's finger to move the activating member; however, the activating member may have configurations other than that shown in the drawings. With reference to FIGS. 2A-2F and 4, various views of the device 100 are shown.

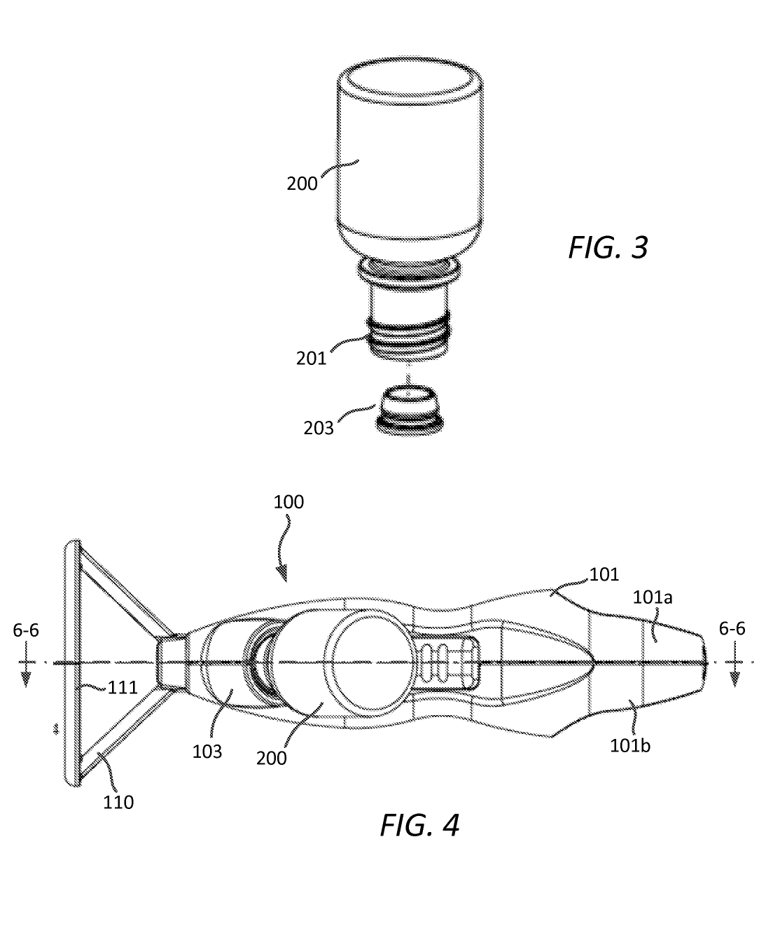

FIG. 3 depicts medicament vial 200 having threaded access members 201 for coupling with mating threads formed on the interior of adapter 103 of housing 101. Other connection mechanisms for forming a fluid tight connection between the vial 200 and the adapter 103 may also be used. In some embodiments the vial may be releasably attached to the dispenser and in other embodiments the vial may be permanently connected to the dispenser. Vial 200 may include or be provided with a pierceable septum 203 that is pierced by spike assembly 405. In other embodiments the vial 200 may comprise an opening into which the spike assembly 405 may be inserted as shown in FIG. 15.

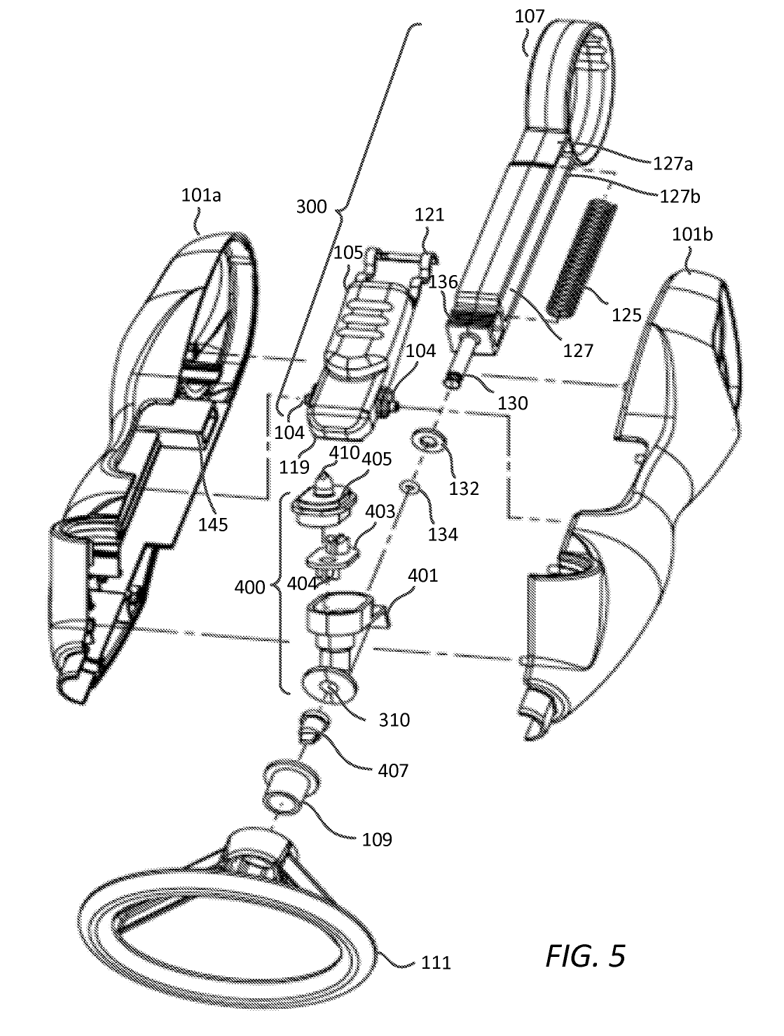

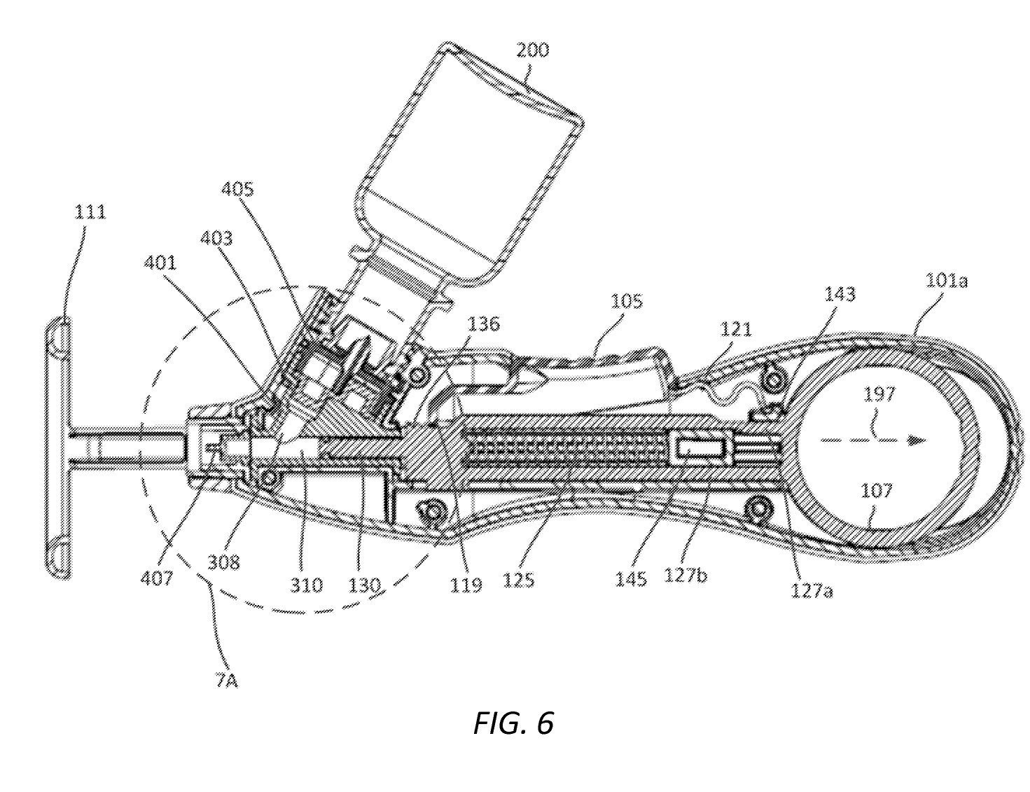

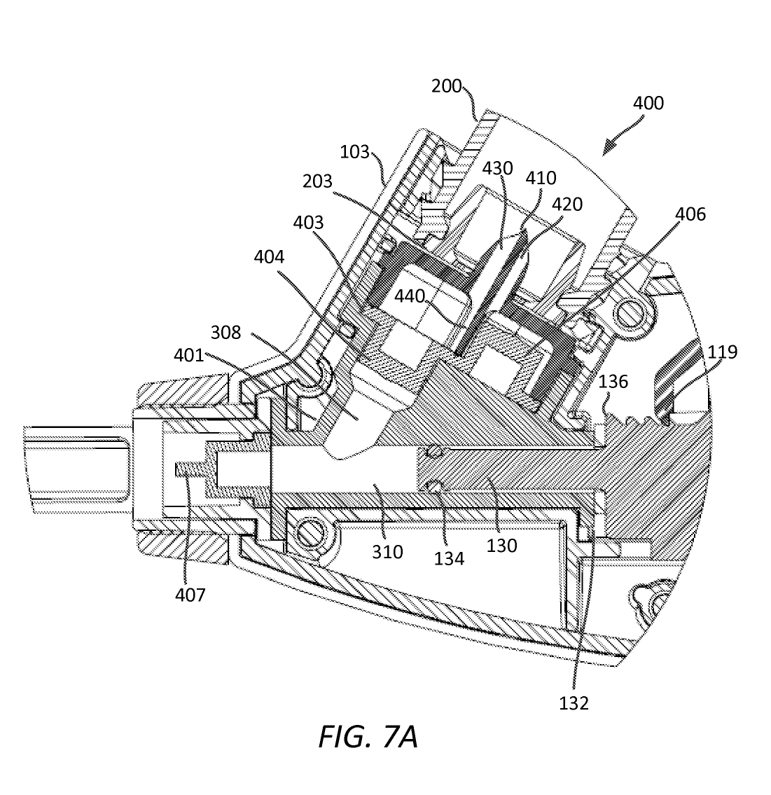

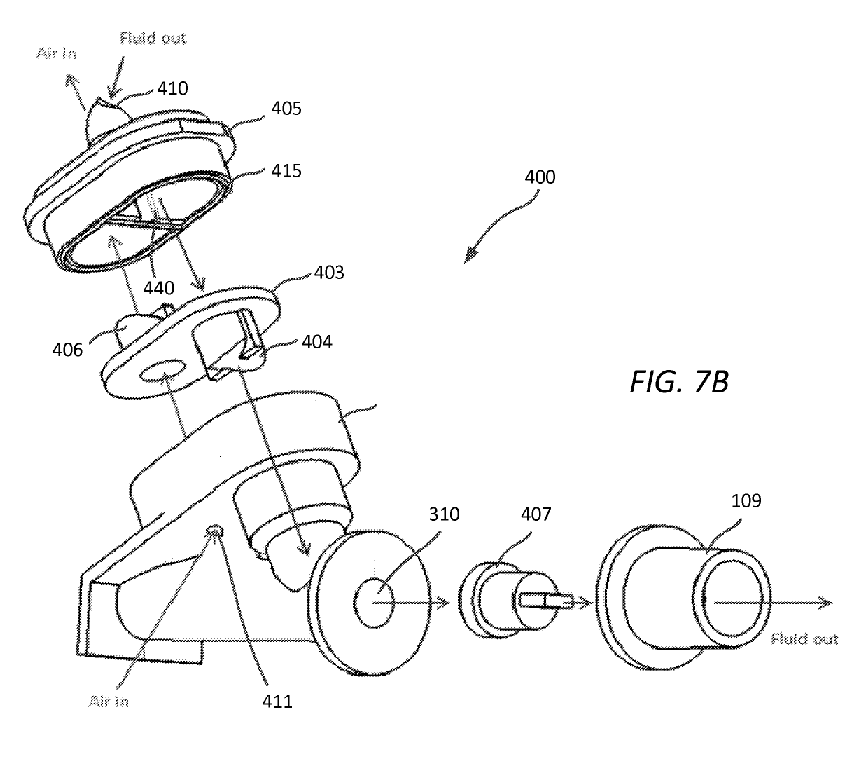

With reference to FIGS. 5 and 6, an exploded view and vertical sectional view of device 100, respectively, are provided showing housing components 101a and 101b that together form housing 101, valve assembly 400, and actuating assembly 300. Valve assembly 400 includes valve housing 401, dual flow control valve member 403 and spike assembly 405. Valve assembly 400 is operably coupled to one-way flow control valve 407 via annular flange 109 such that medicament expelled by valve assembly 400 is delivered to flow control valve 407 under pressure and dispensed from the dispenser 100. Valve assembly 400 includes cylinder 310 formed in valve housing 401 which slidably receives piston 130 of actuator assembly 300. Referring to detailed sectional view FIG. 7A, valve assembly 400 is shown with piston 130 and sealing surface 134 slidably received in cylinder 310. Inlet 308 is fluidically coupled to cylinder 310 and first flow control member 404 controls the flow of fluid into inlet 308. Fluid lumen 430 of spike 410 is fluidically coupled to the first flow control member 404 such that fluid flows from the vial 200 into fluid lumen 430 and through first flow control member 404 to the inlet 308. Spike 410 optionally includes second lumen 420 fluidically coupled to vent 411 (see FIG. 7B) via second flow control member 406. First and second flow control members 404, 406 can be check valves arranged together in opposite flow control arrangement such that medicament from vial 200 is restricted to flowing in one direction from vial 200, through inlet 308 and into cylinder 310, and air is restricted to flow in the opposite direction through vent 411, flow control member 406, lumen 420 to “vent” into vial 200.

Referring now to FIG. 7B, an exploded view of valve assembly 400 is shown. Spike assembly 405 having spike 410 is arranged with flange seal 415 for receiving flow control valve members 404 and 406. Spike assembly 405 has dividing wall member 440 that bifurcates spike 410 into the lumens 420, 430 and isolates the fluid and airflow. Thus airflow is directed through vent 411 through one way flow control valve 406 and lumen 420 whereas medicament flow is controlled through lumen 430 through one way flow control valve 404 through inlet 308 into cylinder 310 in a direction opposite to that of the airflow.

Actuator assembly 300 further includes user-actuator button 105 having pivoting connection points 104 for coupling with housing 101 between distal end 119 and living hinge/biasing mechanism 121. Connection points 104 may comprise pins that engage mating recesses formed in housing components 101a, 101b such that the actuator button 105 rotates about a pivot axis formed by pins 104. One end of the living hinge/biasing mechanism 121 is coupled to member 143 which is secured to housing 101. The living hinge/biasing mechanism 121 biases the distal end 119 of the actuator button 105 into engagement with the user activating member 107 (counterclockwise as viewed in FIG. 6). Distal end 119 of user actuator button 105 is selectively operably coupled to teeth 136 of activating member 107 to hold the activating member 107 in position as will be described. Piston 130 is operatively connected to activating member 107 such that piston 130 moves with the activating member. Piston 130 includes sealing surface 134 (e.g., an O-ring) for providing a liquid tight seal with the cylinder 310. Spacer unit 132 is optionally provided to adjust the stroke of piston 130 in cylinder 310 during use to thereby adjust the amount of fluid delivered upon each activation of the dispenser. Activating member 107 may comprise parallel support members 127a and 127b which provide a space 127 for housing stored energy member 125, depicted as a spring. Housing stop 145 is positioned in space 127 between members 127a and 127b and is fixed in position relative to the housing 101. The stored energy member 125 is trapped between housing stop 145 and wall 127c of the activating member such that the spring is compressed when the activating member is moved in a first direction as represented by arrow 197 of FIG. 6.

During use, activating member 107 is urged rearwardly by user, as shown by directional arrow 197, causing stored energy member 125 to compress against housing stop 145 while drawing piston 130 rearward in cylinder 310. As the piston moves in the first direction rearward in the cylinder 310 fluid is drawn from medicament vial 200 into fluid lumen 430 of spike 410 through one way flow control valve 404 and into cylinder 310. Distal end 119 of actuator button 105 holds the activating member 107 and piston 130 in the retracted state via the engagement of teeth 136 with the distal end 119 of actuator button 105 until the user depresses user actuator button 105. Depression of the actuator button 105 causes the distal end 119 to rotate away from the teeth 136 thereby releasing the stored energy provided by stored energy member 125. Stored energy member 125 moves activating member 107 to move piston 130 in a second direction (opposite to arrow 197) forward in cylinder 310 causing medicament to exit via one way flow control valve 407. In one aspect one way flow control valve 407 is a duck-bill valve with a predetermined cracking pressure of between about 0.2 to about 1.5 pounds. The stored energy member 125 creates a fluid pressure greater than the cracking pressure such that the valve 407 is opened and fluid is dispensed from the dispenser. Other one way flow control valves can be used. The one-way flow control valve 407 provides for an aseptic delivery system. When the actuator button 105 is released the living hinge/biasing member 121 repositions user actuator button 105 to engage end 119 with teeth 136 such that the dispensing cycle may be repeated for additional dispensing. Thus, device 100 provides a “dual-action” mode of operation where the device is first placed in a first state by the user by pulling back on ring activating member 107. Dispensing of medicament requires the user to depress user-actuator button 105 in a separate second action which places the device in a second state whereby the stored energy is released to the valve assembly. Prior to first use, the user may purge the system or may squeeze medicament vial 200 to urge medicament into inlet 308 and/or cylinder 310.

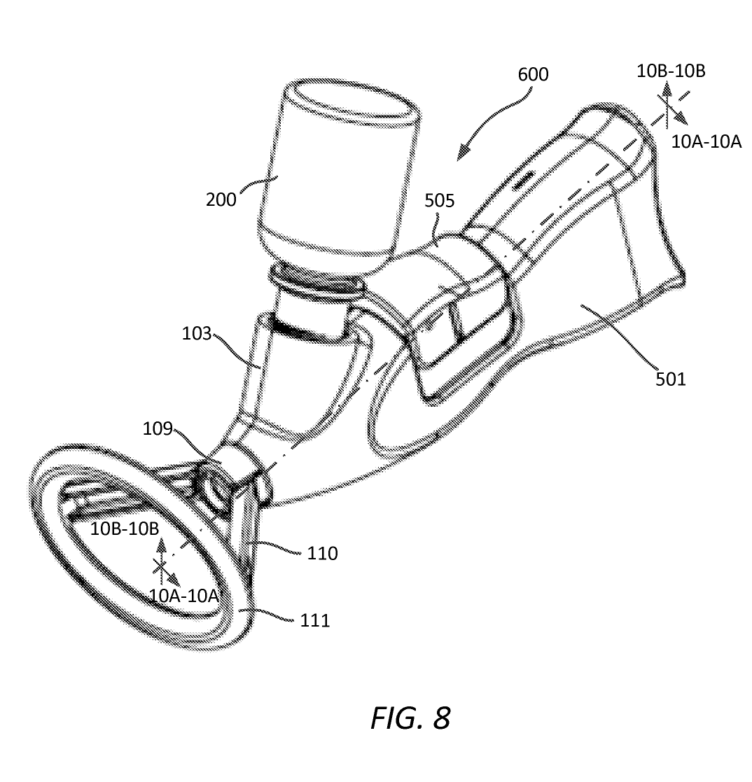

A second embodiment of the device of the present disclosure is shown generally at 600 in FIGS. 8-10C. Device 600 has the identical valve assembly 400 as described with respect to the embodiment of FIGS. 1-7B but uses an alternate actuator assembly 500 as discussed below.

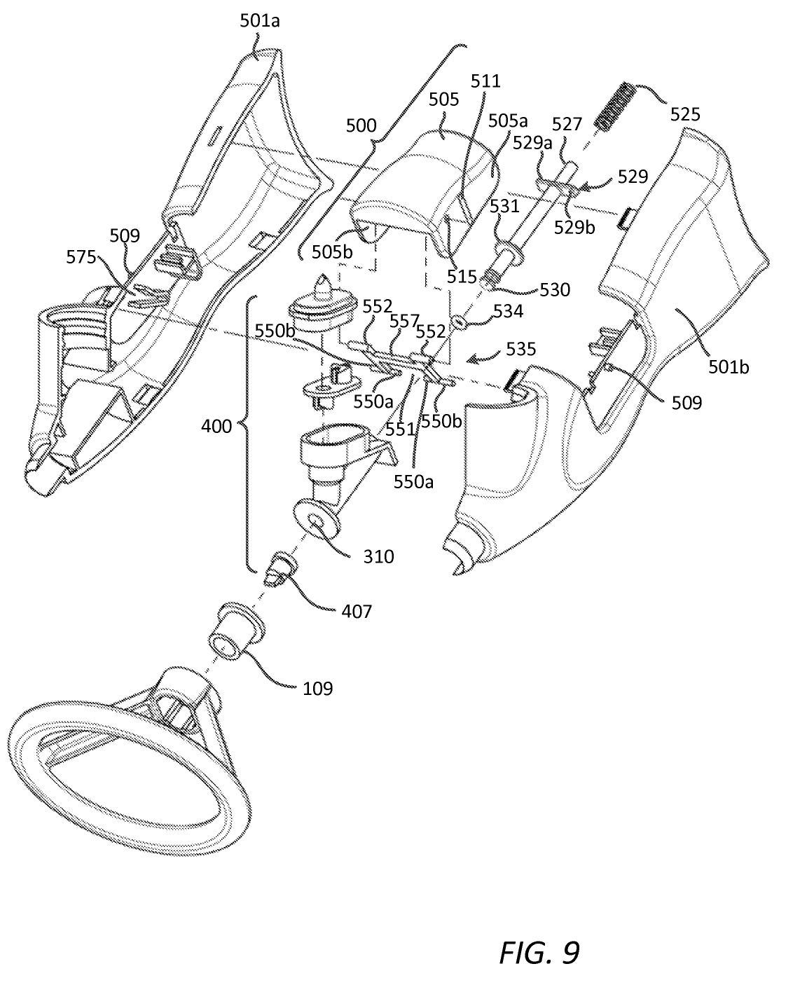

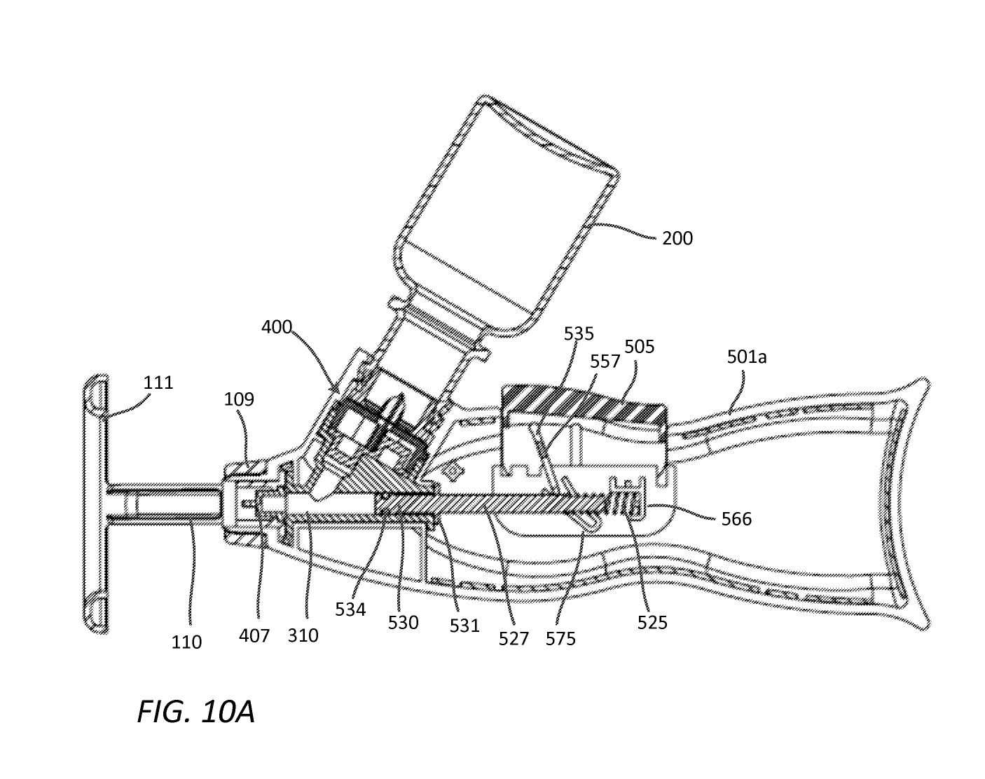

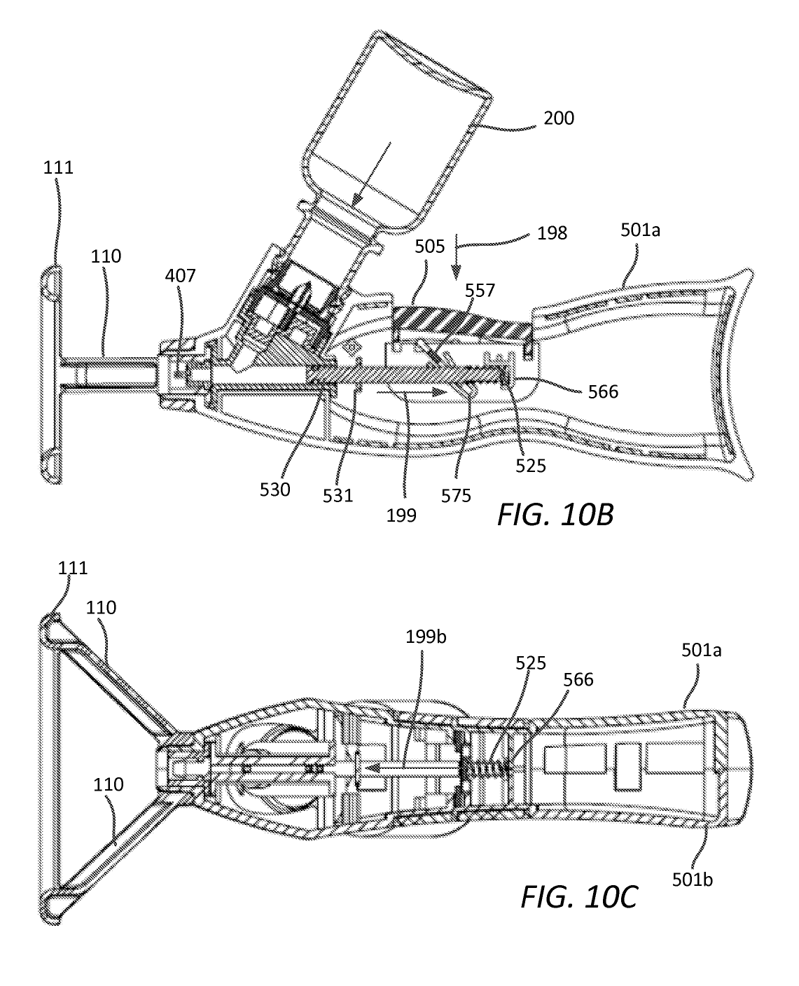

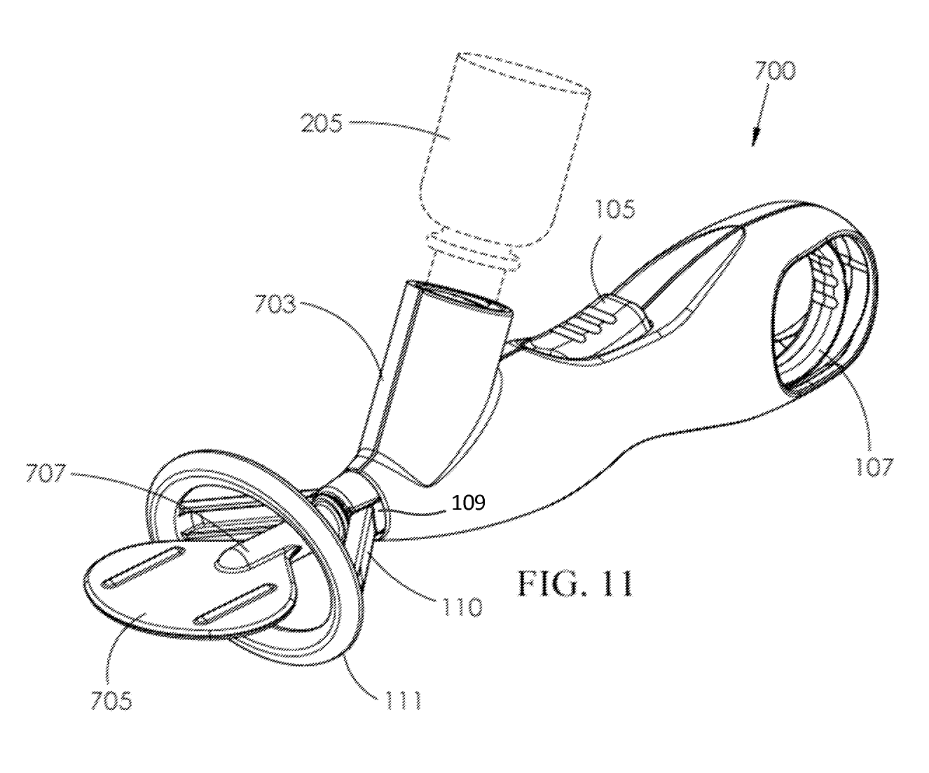

With reference to FIGS. 9, 10A, 10B, and 10C, device 600 is shown with two-piece housing members 501a and 501b that together define housing 501 for containing valve assembly 400 as previously described Device 600 comprises actuator assembly 500 including user actuator button 505 having sidewalls 505a and 505b. Sidewalls 505a, 505b straddle housing members 501a, 501b and actuator button 505 slides on pins 509 that engage mating slotted apertures 511 formed in the sidewalls 505a, 505b to permit the actuator button 505 to be depressed by a user. The sidewalls 505a, 505b further include openings 515 that receive outwardly projecting members 552 of H-like shaped transmission member 535. Outwardly projecting members 552 are positioned on the vertical arms above cross bar 557. Outwardly projecting members 550b and inwardly projecting members 550a are positioned on the vertical arms below cross bar 557. Inwardly projecting members 550a, cross bar 557 and the portion of the vertical arms below the cross bar 557 create space 551 sized to accommodate projecting flange member 529 that extends from activating member or rod 527. Piston 530 is operatively connected to activating member 527 such that piston 530 and activating member 557 move together. Piston 530 includes sealing surface 534 (such as an O-ring) and spacer 531 that limits the length of travel of piston 530 into the cylinder 310. Projecting flange member 529 comprises a rearward face 529a and opposing forward face 529b. Stored energy source 525 (such as a compression spring) is arranged about elongated activating member or rod 527 and butts up against rearward face 529a of projecting flange member 529 and a stop member 566 formed in a fixed position relative to housing 501 such that the spring is compressed between the projecting flange member 529 and the stop member 566 when the activating member 527 is moved in a first direction. Outwardly projecting members 550b of H-like shaped transmission member 535 are operably positioned and slidable in guiding tracks 575 formed in each of housing members 501a, 501b of housing 501. The inwardly projecting members 550a are positioned such that they slidably engage the forward face 529b of projecting flange member 529 when the dispenser is actuated as will be described.

During use, the user depresses user-actuator button 505 as shown by arrow 198 from the position of FIG. 10A to the position of FIG. 10B. The engagement of the actuator button 505 with the outwardly projecting members 552 of the H-like shaped transmission member 535 causes the transmission member 535 to move with the actuator button 505 such that the outwardly projecting members 550b travel in guiding tracks 575 (shown as substantially V-shape) while the inwardly projecting members 550a slidably engage the forward face 529b of projecting flange member 529 of the elongated activating member 527. Because the guiding tracks 575 are angled toward the back of the housing 501 from the top of the tracks toward the bottom of the tracks, the engagement of the inwardly projecting members 550a with the projecting flange member 529 moves the elongated activating member 527 in a first direction rearward as shown by arrow 199. As the activating member 527 is moved rearward, stored energy is created in stored energy member 525. In one embodiment, a spring 525 is compressed between the projecting flange member 529 and the stop 566. Simultaneously, piston 530 is drawn backwards in cylinder 310 of valve assembly 400 causing medicament to be drawn into cylinder 310 and charged for delivery as discussed above. The inwardly projecting members 550a slide down the forward face 529b of projecting flange member 529 as the actuator button 505 is continued to be depressed in the same direction (arrow 198) and the activating member 527 is retracted until the inwardly projecting members 550 reach and move past the lower edge of forward face 529b. The projecting flange member 529 enters space 551 in H-like shaped transmission member 535 such that the activating member 527 is free to move in a second direction (arrow 199b) to the extended position. The stored energy is released from stored energy member 525 to drive elongated activating member 527 and piston 530 forward into the cylinder 310 of valve assembly 400 as shown by arrow 199b (FIG. 10C) so as to release medicament as described above by providing sufficient force to overcome the cracking pressure of valve 407. Thus, FIG. 10C shows device 600 as it passes thru a second state and returns to a first state in essentially a continuous manner.

Thus, device 600 provides a “single-action” mode of operation where dispensing of medicament requires user to depress user-actuator button 505 in a single direction which concurrently retracts the piston 530, provides the stored energy and then releases the stored energy to move the piston into the valve assembly.

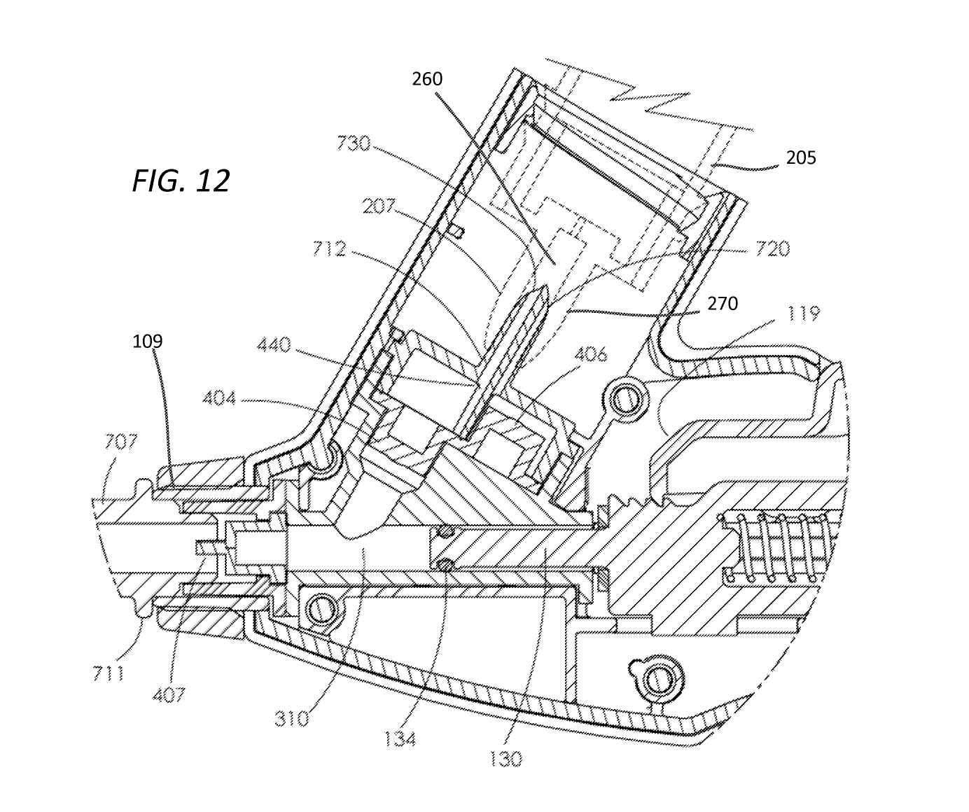

With reference to FIGS. 11 and 12, device 700 provides an alternate valve assembly 400a essentially similar to that of valve assembly 400 discussed previously. Spike 410a is elongated so as to enter opening 260 of vial/container having a tapered dispensing member 270 and provide fluid communication with lumens of spike 720. Device 700 further comprises cover member 750 with elongated member 707 that mates with opening of flange 109. Mating can be via interference fit and the insertion of cover member 707 can be limited by flange 711 of cover member 750. Cover member 750 provides sterility and prevents or eliminates drying of medicament before or in-between uses.

Anti-microbial material can be added to the molded components of the eye device to impart microbial control. Furthermore, while certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures, but may also encompass combinations of elements of the various illustrated embodiments and aspects thereof.

1. A dispensing device, comprising:
a valve assembly comprising a cylinder in fluid communication with a one-way valve and an inlet in fluid communication with the cylinder, the valve assembly further comprising: a spike with at least one lumen; and at least one flow control member for delivering medicament from a medicament container to the inlet;
a piston configured to draw an amount of fluid through the flow control member and inlet into the cylinder upon movement of the piston in a first direction, and to expel the amount of liquid through the one-way valve upon movement of the piston in a second direction; and
an actuator assembly coupled to the piston for moving the piston in the first direction to a first state and for storing energy in a stored energy source and for releasing the stored energy to move the piston in the second direction.

2. The dispensing device of claim 1, wherein the flow control member provides one-way fluidic communication between the at least one lumen and the inlet.

3. The dispensing device of claim 1, wherein the spike has a second lumen fluidically coupled to a second flow control member providing one-way fluidic communication to a vent.

4. The dispensing device of claim 1, wherein the stored energy source comprises a spring.

5. The dispensing device of claim 1, wherein movement of the piston in the first direction delivers a predetermined amount of medicament to the cylinder.

6. The dispensing device of claim 1, wherein movement of the piston in the first direction and the second direction is sequential.

7. The dispensing device of claim 1, wherein the actuator assembly includes an actuator operatively connected to an activating member by a transmission member, the activating member being operatively connected to the piston for moving the piston in the first direction when the actuator is moved in a third direction.

8. The dispensing device claim 7, wherein the transmission member automatically releases the activating member upon continued movement of the activator in the third direction.

9. The dispensing device of claim 1, wherein the one-way valve has a cracking pressure that is less than the force generated by the piston driven by the stored energy source.

10. The dispensing device of claim 1, wherein the spike pierces a penetrable septum of the medicament container.

11. A dispensing device, comprising:
a valve assembly comprising a cylinder in fluid communication with a one-way valve and an inlet in fluid communication with the cylinder, the valve assembly further comprising a spike with a lumen and a flow control member for delivering medicament from a medicament container to the inlet;
a piston configured to draw an amount of fluid through the flow control member and inlet into the cylinder upon movement of the piston in a first direction, and to expel the amount of liquid through the one-way valve upon movement of the piston in a second direction; and
an actuator assembly coupled to the piston for moving the piston in the first direction to a first state and for storing energy in a stored energy source, an actuator for holding the piston in the first state and for releasing the piston to release the stored energy to move the piston in the second direction.

12. The dispensing device of claim 11, wherein the flow control member provides one-way fluidic communication between the lumen and the inlet.

13. The dispensing device of claim 11, wherein the spike has a second lumen fluidically coupled to a second flow control member providing one-way fluidic communication to a vent.

14. The dispensing device of claim 11, wherein the stored energy source comprises a spring.

15. The dispensing device of claim 11, wherein movement of the piston in the first direction delivers a predetermined amount of medicament to the cylinder.

16. The dispensing device of claim 11, wherein movement of the piston in the first direction and the second direction is sequential.

17. The dispensing device of claim 11, wherein the actuator assembly includes an activating member for moving the piston in the first direction.

18. The dispensing device claim 17, wherein the actuator engages the activating member to hold the activating member in the first state.

19. The dispensing device of claim 11, wherein the one-way valve has a cracking pressure that is less than the force generated by the piston driven by the stored energy source.

20. The dispensing device of claim 11, wherein the spike pierces a penetrable septum of the medicament container.

21. (canceled)

22. A method of dispensing medicament into the eye of a mammal, the method comprising:
providing a dispensing device, the dispensing device comprising a valve assembly, the valve assembly comprising a cylinder in fluid communication with a one-way valve and an inlet in fluid communication with the cylinder, the valve assembly further comprising a piston, a spike having at least one lumen and at least one flow control member for delivering medicament from a medicament container to the inlet, wherein:
the piston is operable in a first direction to draw an amount of fluid through the flow control member and inlet into the cylinder and for storing energy in a stored energy source, and
the piston is operable for releasing the stored energy so as to move the piston in a second direction to expel the amount of fluid through the one-way valve for dispensing the amount of fluid into the eye of a mammal.
